(19) United States
(12) Patent Application Publication
(10) Pub. No.: US 2019/0300218 A1
(43) Pub. Date: Oct. 3, 2019

(54) Method and Filling Machine for Filling Packages Open on One Side via an Elongated Nozzle Slot

(71) Applicant: SIG Technology AG, Neuhausen am Rheinfall (CH)

(72) Inventors: Thomas Patzer, Juelich (DE); Christian Weiler, Geldern (DE)

(21) Appl. No.: 16/464,861

(22) PCT Filed: Oct. 19, 2017
(86) PCT No.: PCT/EP2017/076744

§ 371 Date: May 29, 2019

(30) Foreign Application Priority Data
Nov. 30, 2016 (DE) 10 2016 123 142.8

(51) Int. Cl. B65B 55/10; B65B 55/06; B65B 39/12; B65B 39/14; A61L 2/18; A61L 2/07; A61L 2/20; A61L 2/26

(52) U.S. Cl. CPC B65B 55/10; B65B 55/06; B65B 39/12; B65B 39/145; A61L 2/186; A61L 2202/182; A61L 2/208; A61L 2/26; A61L 2202/15; A61L 2202/23; A61L 2/07

(57) ABSTRACT

A method is described and illustrated for filling packages open on one side, in particular cardboard composite packages, with flowable products, in particular foodstuffs, in a filling machine. So that the packages can be impinged sufficiently and reliably with hot air, drying air and/or sterilising agent simply, quickly and with a small space requirement, it is provided that hot air, drying air and/or sterilising agent is introduced into the packages 2 open on one side from above via at least one nozzle as they are transported past under the nozzle and in that the hot air, the drying air and/or the sterilising agent are directed towards the packages from at least one elongated nozzle slot extending transverse to the transport direction.

The invention relates to a method for filling packages open on one side, in particular cardboard composite packages, with flowable products, in particular foodstuffs, in a filling machine. Furthermore, the invention relates to a filling machine for filling packages open on one side, in particular cardboard composite packages, with flowable products, in particular foodstuffs, preferably according to the method specified, wherein at least one nozzle is provided for introducing hot air, drying air and/or sterilising agent from above into the packages open on one side and wherein a transport device is provided for transporting the packages open on one side through the filling device and under the at least one nozzle.

Different filling machines for filling packages open on one side with products, especially in the form of foodstuffs, are already known. The packages are preferably filled with flowable foodstuffs. The packages used in particular are packages which are open at an upper side in order to provide an opening for filling the packages. The packages can be, for example, cardboard composite packages which are formed from a packaging material in the form of a laminate comprising a cardboard layer and outer, in particular thermoplastic, plastic layers, for example made of polyethylene (PE). The carton provides the packages with sufficient stability to allow the packages to be easily handled and stacked, for example. The plastic layers protect the cardboard from moisture and allow sealing of the packaging material to form a sealed package. In addition, further layers, such as an aluminium layer, may be provided to prevent diffusion of oxygen and other gases through the package.

The filling of the packages with foodstuffs is typically performed under sterile conditions. Not only the foodstuffs to be but also the package has to be sterilised. To effect this, the foodstuffs are usually heated for a certain period of time. First, the packages are generally purged with sterile hot air. A sterilising agent is then introduced into the packages heated thus, which typically is or contains hydrogen peroxide. As the package is preheated, high sterilisation reaction rates are achieved, avoiding the formation of excess condensate in the package. Moisture and residual hydrogen peroxide is then removed from the package by drying the package with preferably hot and sterile drying air. The filling of the sterile package then takes place.

The devices for introducing hot air, drying air and/or sterilising agent comprise nozzles for targeted impinging of the packages open on one side with the appropriate medium. During impinging of the packages with hot air, drying air, and/or sterilising agent, the packages may be transported past under the respective nozzles, which typically have round nozzle outlets. The transport of the packages can be effected continuously, if necessary, at a constant speed. If desired, the nozzles may be moved with the packages, such that there is no relative velocity between the packages and the nozzles. However, it is often simpler and more cost-effective if the nozzles are provided in a stationary position. In any case, it must be ensured that the packages are sufficiently impinged with hot air, drying air, and/or sterilising agent, in order that they are sufficiently preheated, dried, and/or sterilised. On the one hand, sufficient hot air, drying air and/or sterilising agent must be directed from the nozzle onto or into the packages and the package must remain under the nozzle for a sufficiently long time. At the same time, however, filling machines are intended to fill as many packages as possible in the shortest possible time and in the smallest possible space. It is therefore the object of the present invention to design and further develop the method and the filling machine of the type mentioned and explained above, in such a way that the packages are reliably and sufficiently impinged with hot air, drying air and/or sterilising agent in a simple and fast manner and with minimal space required.

This object is achieved according to claim 1 by a method for filling packages open on one side, in particular cardboard composite packages, with flowable products, in particular foodstuffs, in a filling machine,
in which hot air, drying air and/or sterilising agent is introduced from above via at least one nozzle into the packages open on one side, which are transported past under the nozzle,
in which the hot air, the drying air and/or the sterilising agent is directed from at least one elongated nozzle slot, extending transverse to the transport direction towards the packages.

The above object is also achieved in a filling machine with the preamble of claim 8, in that the at least one nozzle has at least one elongated nozzle slot, extending transversely to the transport direction, for directing the hot air, the drying air and/or the sterilising agent towards the packages open on one side.

The invention has recognised that the processing of the packages open on one side with hot air, drying air and/or sterilising agent can be improved if the appropriate medium is applied via a nozzle having an elongated nozzle slot extending transversely to the transport direction. In this way, the corresponding package can be impinged in the same time with more medium, without a major adaptation of the filling machine being required. In addition, the introduction of the appropriate medium into the packages is improved in such a way that the inner surfaces of the packages more reliably come into contact with the appropriate medium in the desired manner. The packages are thus heated, dried and/or sterilised more uniformly and as intended. In addition, this has the advantage that the processing of the packages with the appropriate medium is less dependent on the type and dimensions of the packages. Thus, the filling machine can be converted for operation with other types of packages without much change in terms of the design or process management.

In the present case, the term nozzle is preferably understood very generally. A nozzle with significant cross-sectional constriction for producing a significant pressure loss or a greatly widening free jet is not necessarily required. The nozzle is merely intended to allow a reliable and targeted feeding of the appropriate medium along the length of the nozzle. In a particularly simple case, the nozzle can be configured, for example, as a hollow component which tapers off and ends in an elongated, in particular rectangular, cross section.

Furthermore, the terms ‘hot air’ and ‘drying air’ are preferably to be understood very broadly. The appropriate medium is used for preheating and drying, which could also be done entirely without air, but with another gas. Thus, when the terms ‘hot air’ and/or ‘drying air’ are used, the term ‘air’ is preferably understood generally as a gas or mixture of gases. However, since air is readily available at low cost, it is particularly preferable if actual air is used. Even in practice, air is generally used. Therefore, in the present case for the sake of clarity and to avoid unnecessary repetition, the terms ‘hot air’ and ‘drying air’ are used, even if these media need not be limited to air and could refer in principle to ‘heating medium’ or ‘drying medium’. The air or another medium is preferably sterile in order to avoid contamination of the packages. For sterilisation, the air or the appropriate medium can be passed through a correspondingly fine filter, which separates even the smallest solids.

The packages are preferably laminate packages formed from packaging laminates. In a particularly preferred case, these are cardboard composite packages made of a packing laminate comprising at least one cardboard layer, preferably at least one barrier layer, such as aluminium, polyamide and/or an ethylene-vinyl alcohol, and outer layers made of a thermoplastic material, in particular polyethylene (PE). The longitudinal edges of a packaging material blank formed from the packaging material laminate are sealed together to form what is known as a packaging sleeve. One of the open ends of the packaging sleeve can be folded together and sealed to form a package base. This is often referred to as a packaging cup or a package open on one side. After filling, the still open end of the packaging sleeve can also be closed by folding and sealing into a packaging top. This is then often referred to as a package.

For ease of understanding and to avoid unnecessary repetition, the method and the filling machine for filling packages open on one side will be described together below, without distinguishing in detail between the method and the filling machine. However, from the context it will be apparent to those skilled in the art which particular feature is preferred with respect to the method and the filling machine.

In a first particularly preferred method, the hot air, the drying air and/or the sterilising agent is directed towards the packages open on one side via an elongated nozzle slot, which extends transverse to the transport direction, over at least substantially the entire width of the packages transported past underneath. If necessary, the packages can be decelerated or stopped in the region of the nozzle slot for a short time. In this way, the entire width of the packages can be utilised for introducing the appropriate medium. This not only leads to a more uniform distribution of the appropriate medium in the package, but also leads to the fact that at a given transport speed of the packages more medium can be easily directed into the packages, without the need for increased equipment complexity or a larger filling machine.

Alternatively or additionally, the nozzle slot may extend at least substantially obliquely to the transport direction of the packages in order to increase the exposure time of the appropriate medium to the package for a given transport speed of the packages. In other words, the residence time under the corresponding nozzle is thus increased without having to stop the package under the nozzle. In doing so, the introduction location of the appropriate medium into the package changes while the packages are transported past. Due to the distribution of the appropriate medium in the package, quite good processing results are however achieved without increased equipment complexity or larger filling machines being required. This is particularly the case when the nozzle slot is not aligned overly in the transport direction of the packages or overly transverse to the transport direction. Good processing results have been found, in particular, when the angle between the nozzle slot and the transport direction is between 25° and 65°, preferably between 35° and 55°, in particular between 41° and 50°.

In particular for processing relatively high packages, it may be expedient if the hot air, drying air and/or the sterilising agent emerges out of the at least one nozzle at least substantially perpendicular to the transport direction of the packages and/or vertically. However, this can also lead to a better distribution of the appropriate medium in the packages and thus to a more comprehensive and reliable processing of the packages, regardless of the height of the packages. High packages are in particular those in which the height is greater than the maximum diagonal of the cross section. In addition, an angle deviation of +/−10°, preferably +/−5°, in particular +/−3°, can be understood to mean essentially perpendicular.

In order to extend the processing time during the impinging of the packages with hot air, drying air and/or sterilising agent without having to lengthen the filling machine accordingly, the flow direction of the hot air, the drying air and/or the sterilising agent as it exits from the nozzle may be adjusted, in particular pivoted, parallel and/or transverse to the transport direction of the packages, while a package is transported past under the nozzle. A targeted impinging of the corresponding inner sides of the packages can be achieved by the injection of the appropriate medium being at least at times oblique, which can be achieved both in the case of an adjustment or of a pivoting parallel as well as transverse to the transport direction. Alternatively or additionally, however, a uniform purging of the packages with the appropriate medium can be achieved in this way.

If the flow direction of the hot air, the drying air and/or the sterilising agent is adjusted, in particular pivoted, towards the transport direction of the packages as it exits the nozzle, while a package is transported past under the nozzle, the processing time with the appropriate medium can be extended. Thus, for example, the appropriate medium can already be directed into the package before the package is actually arranged below the corresponding nozzle. Alternatively or additionally, the appropriate medium can still be directed into the package if the package is no longer arranged under the nozzle.

In order to simplify the process management and to reduce the equipment complexity, the packages can be transported past the at least one nozzle continuously, in particular at a constant speed. However, it may also be expedient for the treatment of the packages if the packages are briefly decelerated or even stopped for a short time while being transported past the at least one nozzle. Ultimately, if necessary, a timed or continuous transport of the packages, if necessary with a varying transport speed, is possible.

In particular, when the packages are transported continuously past the at least one nozzle, it is conceivable that the packages are also impinged with the appropriate medium on their outer side surfaces. This is of particular advantage in particular in connection with the sterilising agent, since in this way it is also possible to partially sterilise the outer sides of the package, in particular in the region of the subsequent packing gable.

In a first particularly preferred embodiment of the filling machine, the nozzle slot is arranged transversely to the transport direction at least substantially over the entire width of the packages transported past underneath. Under certain circumstances, however, it may also be sufficient if the nozzle slot extends only over 80% of the width of the package transported past underneath. In addition, an extension of the nozzle slot over up to 120% of the width of the package will be sufficient in many cases. However, special applications are conceivable in which a length of the nozzle slot up to 150%, up to 200% or even up to 300% of the width of the packages can lead to expedient processing of the packages. The nozzle slot can be extended in a manner corresponding to the width of the packages, whereby the package can be more reliably and more uniformly purged with the appropriate medium. In this case, the width of the packages is preferably understood to mean the length of the packages perpendicular to the longitudinal axis of the package or the vertical axis of the package, as well as perpendicular to the transport direction of the packages. By the appropriate arrangement of the nozzle slot, ultimately a more homogeneous treatment of the packages is achieved by the appropriate medium. This is especially true in the case where the nozzle slot is approximately as wide or slightly wider than the package. Alternatively, for the same reason, the nozzle slot may be slightly longer than the maximum diagonal of the cross section of the opening of the package.

If the nozzle slot is alternatively or additionally arranged obliquely to the transport direction of the packages, the treatment time with the appropriate medium or the residence time under the corresponding nozzle can be extended. This is particularly the case when the angle between the nozzle slot and the transport direction of the packages is between 25° and 65°, preferably between 35° and 55°, in particular between 41° and 50°.

If this leads to favourable results, the nozzle slot may be formed obliquely to the transport direction and/or elongated, in particular in the transport direction, so that the nozzle slot is at least temporarily arranged over two packages successively transported past the nozzle. This can be favourable for the processing of the packages but also leads to an additional need for the medium to be dispensed via the nozzle.

If the nozzle is configured to direct the hot air, the drying air and/or the sterilising agent at least substantially perpendicular to the transport direction of the packages and/or vertically in the direction of the packages open on one side, the packages, in particular high packages, can be treated more reliably. In particular, the base region of packages can be sufficiently treated with short processing times.

The processing of the packages can be made more reliable or at least more rapid if the at least one nozzle is suitably designed such that, preferably by an adjustment of the nozzle, in particular by a pivoting of the nozzle, it displaces, in particular pivots, the flow direction of the hot air, the drying air and/or the sterilising agent as it exits the nozzle parallel to the transport direction of the packages, in the transport direction of the packages and/or transverse to the transport direction of the packages, while a package is transported past under the nozzle. Thus, a medium can be introduced in a targeted manner laterally into the packages and thus specifically against certain lateral inner surfaces of the packages and/or into the package at times when the packages are not yet and/or are no longer arranged below the corresponding nozzle. This is realised in a simple manner in that the nozzle and/or the nozzle slot is adjusted or pivoted. Alternatively or additionally, at least one guide device, for example in the form of a guide plate, may be provided which contributes to the desired adjustment of the flow direction of the corresponding medium by suitable adjustment, in particular pivoting, while a package to be treated with the corresponding medium is transported past under the corresponding nozzle.

For the targeted, precise and reliable transport of the packages through the filling machine, a transport device for the sake of simplicity may be provided, having cells and/or carriers for receiving the packages. The packages can then be transported with the carriers or the cells through the filling machine or past the corresponding nozzle, without causing damage to the packages.

Alternatively or additionally, the transport device can be designed to transport the packages open on one side continuously, in particular at a constant speed, past under the at least one nozzle. It is particularly structurally simple if the packages are transported along a straight line. Thus, the complexity of the transport device is reduced, which contributes to a reduction in costs.

The invention will subsequently be explained in more detail with reference to a drawing depicting only exemplary embodiments. The following are shown in the drawings:

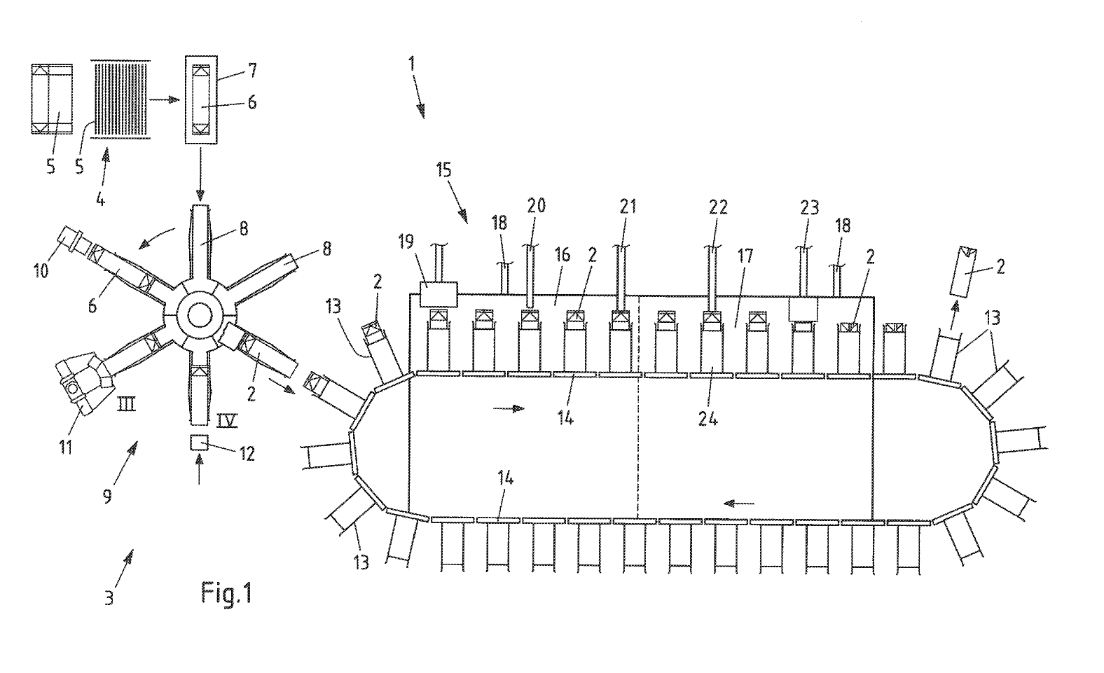

FIG. 1 A filling machine according to the invention for carrying out the method according to the invention in a schematic side view,

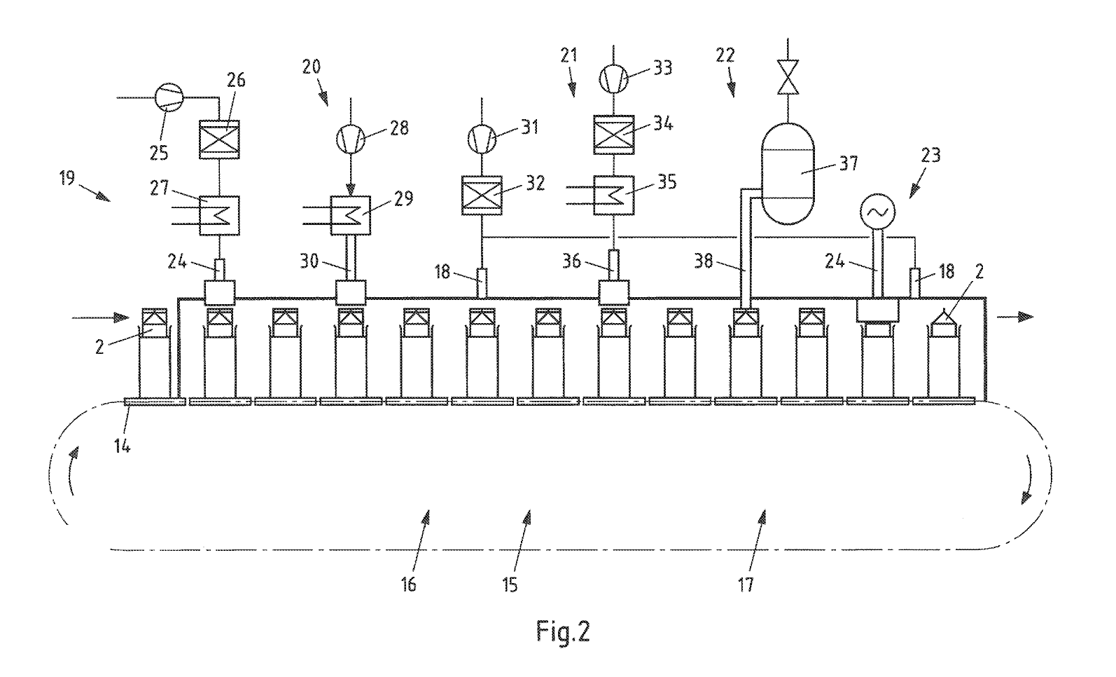

FIG. 2 A detail of the filling machine in a schematic side view,

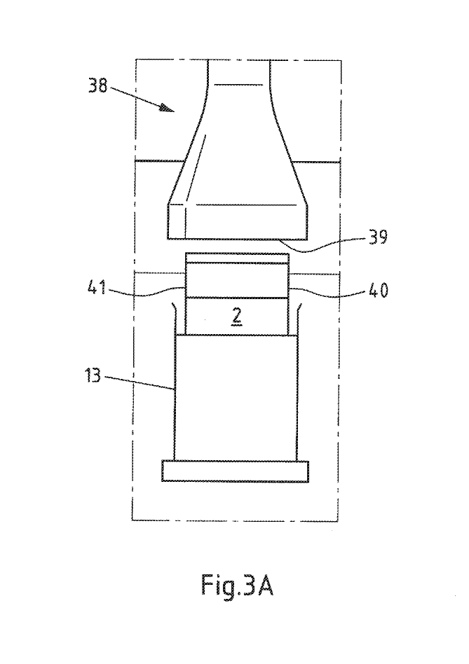

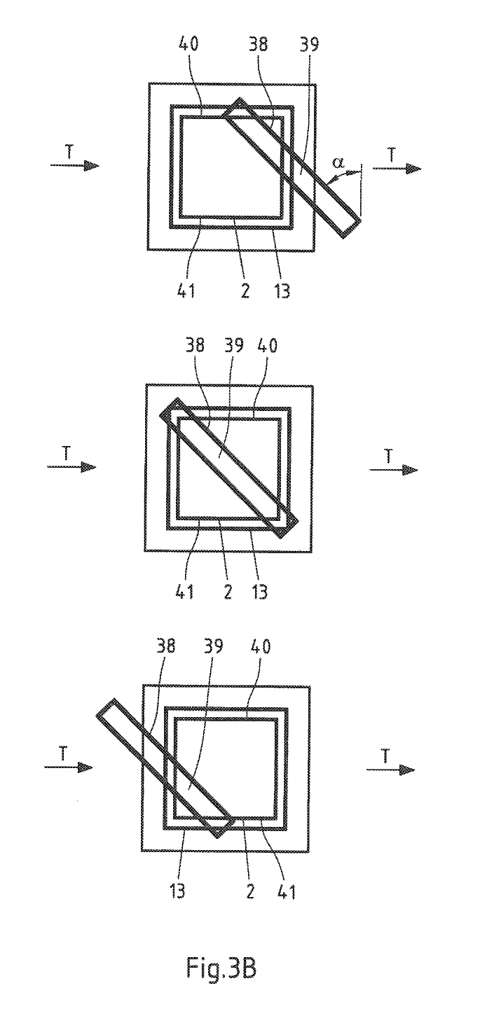

FIG. 3A-B The introduction of hot air, drying air and/or sterilising agent into a package in a schematic side view and a schematic plan view at three different times and

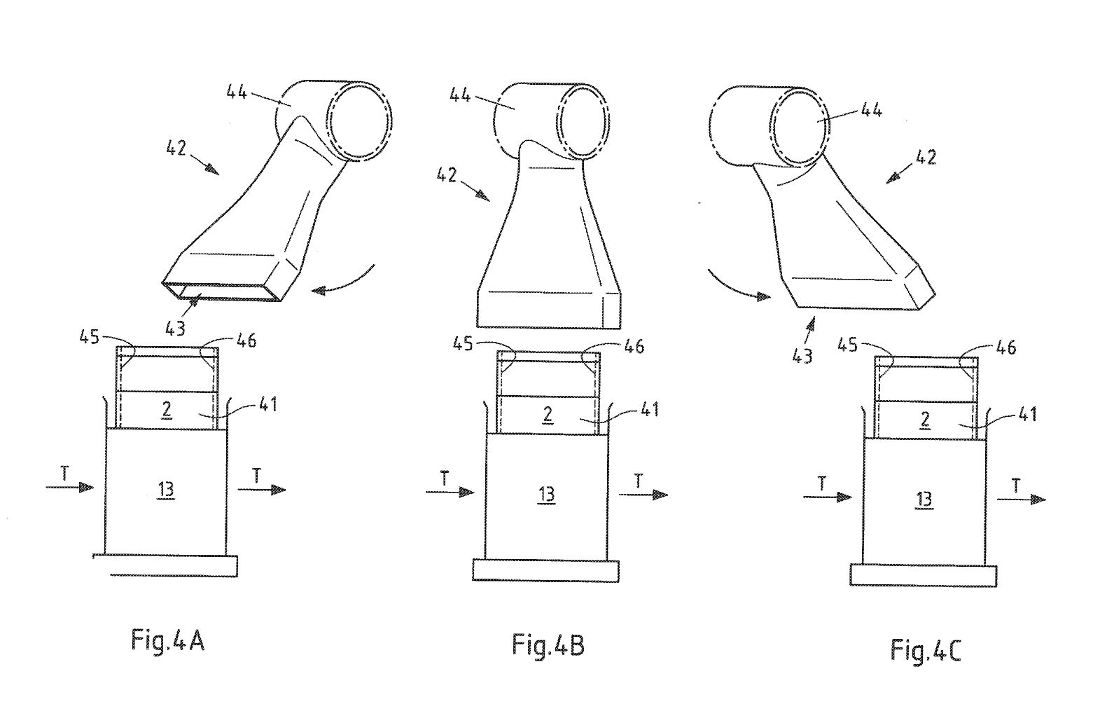

FIGS. 4A-C An alternative introduction of hot air, drying air and/or sterilising agent into a package in a schematic side view at three different times.

FIG. 1 shows a filling machine 1 for filling packages 2, in particular with flowable foodstuffs, which in this case comprises a device 3 for shaping the packages 2 to be filled. In principle, however, it is also possible for the filling machine to be supplied with packages 2 open on one side, already prepared to be filled. The illustrated and in this respect preferred filling machine 1 also has a number of parallel processing lines, of which only one processing line is shown in FIG. 1. Each processing line is associated with a bundle 4 of package blanks 5, whose longitudinal edges are sealed to each other and so form packaging sleeves 6 open on both sides. By a feed device 7, the packaging sleeves 6 are unfolded and pushed onto a mandrel 8 of a mandrel wheel 9.

The mandrel 9 is cyclically, that is incrementally, rotated counterclockwise. The packaging sleeves 6 are processed in different positions. First, an edge is heated with a heating unit 10 using hot air and then pre-folded in a prefold 11 to then seal the prefolded edge to form a base using a press 12. Thus a package 2 is obtained, which is open at one end and sealed at the other end and is transferred to a cell 13 of a transport device 14. In the illustrated and in this respect preferred filling machine 1, the cells are transported continuously, i.e. without stopping in specific positions, through the filling machines. If necessary, the cells are thereby accelerated and/or decelerated or the transport of the cells through the filling machine 1 is carried out at a constant speed.

The package 2 is transported by means of the transport device 14 through an aseptic chamber 15, which is successively subdivided into a sterilisation zone 16 and a filling and sealing zone 17. The transport of the packages 2 does not have to be straight, but can also take place in at least one arc or even in a circle, depending on whether the filling machine 1 is an inline filling machine or a rotary unit. The aseptic chamber 15 is rinsed with sterile air via corresponding sterile air connections 18 in order to prevent the entry of germs.

Sterile hot air is first blown into the open-topped packages 2 by a preheating device 19 in order to preheat the packages 2. Subsequently, in a sterilising device 20, a sterilising agent is injected into the packages, which reacts more strongly in the preheated packages 2 and condenses less. Hydrogen peroxide is particularly suitable as a sterilising agent. The hydrogen peroxide may be introduced together with steam and/or air into the package 2 open on one side. After sterilising the packages 2, the interior of the packages 2 is dried in a drying device 21 by impinging with drying air. The drying air is preferably hot and sterile.

After passing from the sterilisation zone 16 into the filling and sealing zone 17, the packages open on one side 2 are positioned below a filling device 22 and filled with a foodstuff. The filled packages 2 are then closed with a closing device 23 by folding the upper open area of the package 2 and sealing it. The sealed packages 2 are then removed from the cells 13 of the transport device 14 and further processed if needed. The now empty cells 13 of the transport device 14 are moved further towards the mandrel wheel 9 in order to receive more packages 2 there.

In FIG. 1, the preheating device 19, sterilising device 20, drying device 21 and filling device 22 are shown only schematically for the sake of better clearness and clarity. Therefore, in FIG. 2, the aseptic chamber 15 is shown again in detail. The illustration of FIG. 2 is also very schematic, but in particular the nozzles of the preheating device 19, sterilising device 20 and drying device 21, with which the hot air, the drying air or the sterilising agent is directed towards the packages, are shown larger.

In the aseptic chamber 15 of FIG. 2, a preheating device 19, which has a nozzle 24 for introducing hot air into the packages 2, is initially provided in the sterilisation zone 16. The hot air is suctioned in the illustrated and in this respect preferred filling machine 1 via a compressor 25 from the environment and filtered by means of a filter 26, in order to separate microorganisms and so to sterilise the air. Subsequently, the sterile air is heated in a heating device 27. The packages 2 open on one side are transported past continuously under the stationary nozzle 24 of the preheating device 19.

Thereafter, the packages 2 arrive in a sterilising device 20, in which a sterilising agent, which is an aqueous hydrogen peroxide solution in the illustrated and in this respect preferred filling machine 1, is supplied via a pump 28 and vaporised in a vaporiser 29. The mixture of hydrogen peroxide and steam, which still can be supplied with air, is introduced via a nozzle 30 into the packages 2 transported past underneath.

In the area downstream of the sterilising device 20, sterile air is directed via sterile air connections 18 into the sterilisation zone 16, which can alternatively or additionally also take place at other locations of the aseptic chamber 15. For this purpose, air is suctioned in via a compressor 31 from the environment and pressed through a filter 32, in which all microorganisms are separated to sterilise the air. In the subsequent drying device 21, the sterile packages are dried with hot drying air, which is also suctioned in via a compressor 33 and sterilised in a filter 34. The air sterilised thus is heated in a heating device 35 and directed into the packages 2.

The dried packages 2 are then transferred to the filling and sealing zone 17, where the packages 2 are filled with the product in the form of a foodstuff in the next two positions of the transport device 14. The product is supplied in the filling device 22 via a reservoir 37 and is directed into the packages 2 via a nozzle 39. The filled packages 2 are then closed by a closing device 23 by sealing the upper edge and transported out of the aseptic chamber 15.

In the illustrated and in this respect preferred filling machine 1, so called empty positions are provided between individual processing positions along which packages 2 are transported without being processed, while other packages 2 are processed in one of the previously described ways. The arrangement and the number of empty positions can be chosen in different ways, without this being of particular importance. Incidentally, alternative preheating devices 19, sterilisation devices 20, drying devices 21 and/or filling devices 22 may also have additional nozzles in order to process the packages 2 with the corresponding media and/or to fill them with product. Then, the processing and/or filling of the packages 2 is carried out successively with at least two nozzles in order to extend the processing time or to make the processing more reliable.

In FIGS. 3A-B, the introduction of hot air, drying air, sterilising agent or product to be filled is shown in a side view opposite to the transport direction T and in a plan view at three quickly succeeding points in time. The transport direction T of the packages 2 is indicated by arrows. The package 2 is held in a cell 36 of a transport device 14, with which the package 2 is passed continuously under the corresponding nozzle 38, while hot air, drying air or sterilising agent is introduced into the package 2 via the nozzle 38.

The illustrated and in this respect preferred nozzle 38 has an elongated nozzle slot 39 which extends obliquely to the transport direction T of the packages 2 below the nozzle 38, and at an angle of approximately 46°. The elongated nozzle slot 39 extends transversely to the transport direction T over a length which exceeds the width of the packages 2 transversely to the transport direction T. The nozzle slot 39 is also arranged such that the nozzle slot 39 protrudes outwards to both sides of the package 40, 41 transversely with respect to the transport direction T. The middle of the packages 2 is thereby guided at least substantially beneath the centre of the illustrated nozzle slot 39.

The individual views of FIG. 3B illustrate, from left to right, the conditions at three different points in time when a package 2 is being transported past the nozzle 38. Due to the inclination of the nozzle slot 39, the package 2 first arrives under the nozzle slot 39 on its left side of the package 40, as seen in the transport direction T, where the appropriate medium, namely hot air, drying air or sterilising agent, first enters the package 2. The impinging of the packages 2 with the illustrated and in this respect preferred nozzle 38 takes place at least substantially in the vertical direction. A short time later, the nozzle slot 39 extends over the entire width of the package 2 and therefore is applied with the appropriate medium over the entire width of the package 2. Somewhat later, the left side of the package 40 has already passed completely under the nozzle slot 39, while the right side of the package 2, as seen in the transport direction T, is still located below the nozzle slot 39 and is therefore still impinged with the appropriate medium.

The appropriate medium can flow out continuously from the at least one nozzle slot 39. This is particularly simple and leads to a reliable process. However, this tends to consume more medium than would be absolutely necessary for the treatment or processing of the packages 2. To reduce this excess, the spacing of the packages 2 when passing under the at least one nozzle 38 can be reduced.

In FIGS. 4A-C, alternative impinging of the packages 2 with hot air, drying air or sterilising agent by an alternative nozzle 42 is shown in a side view at three different times. The peculiarity of the nozzle 42 is that the nozzle 42 or the elongated nozzle slot 43 is provided to be pivotable via an adjustment device 44 not shown in detail. First, as shown in FIG. 4A, the nozzle 42 is inclined obliquely with respect to the transport direction T in order to introduce the medium into the package 2 at a time when the package 2 is not yet arranged below the nozzle 42 and to more intensively process a the back of a package 45 downstream in the transport direction. As the package 2 is transported further in the transport direction T, the nozzle 42 also pivots in the transport direction T of the package 2. At a later time, in which the package 2, as shown in FIG. 4B, is provided approximately centrally below the nozzle 42, the nozzle 40 is accordingly directed approximately vertically downwards, in order to inject the medium comprehensively into the package 2 open on one side. Thereafter, the package 2 again moves away from the nozzle 42, wherein the nozzle 42 continues to pivot with the package 2 in its transport direction T. Thus, the time is reached when the package 2, as shown in FIG. 4C, has already been transported away from the nozzle 42, wherein the nozzle 42 is however still administering medium into the package 2 due to its inclination, and in particular directs against the front side 46 of the package 2 in the transport direction. Before the next package 2, as shown in FIG. 4A, approaches the nozzle 42, the nozzle 42 is pivoted against the transport direction T of the packages 2 back to a starting position, in order to then be pivoted again with the transport direction T of the packages 2 into an end position.

The pivoting of the nozzle 42 could alternatively or additionally also take place transversely to the transport direction T. Likewise, the nozzle 42 could be stationary and not adjustable, but the flow direction of the medium could be deflected by a movable flow guide into the respectively desired direction.

### LIST OF REFERENCE NUMERALS

1 Filling machine

2 Package

3 Device

4 Bundle

5 Package blank

6 Packaging sleeves

7 Feed device

8 Mandrel

9 Mandrel wheel

10 Heating unit

11 Pre-folding

12 Press

13 Cells

14 Transport device

15 Aseptic chamber

16 Sterilisation zone

17 Filling and sealing zone

18 Sterile air connection

19 Preheating device

20 Sterilising device

21 Drying device

22 Filling device

23 Closing device

24 Nozzle

25 Compressor

26 Filter

27 Heating device

28 Pump

29 Vaporiser

30 Nozzle

31 Compressor

32 Filter

33 Compressor

34 Filter

35 Heating device

36 Cell

37 Reservoir

38 Nozzle

39 Nozzle slot

40 Package side

41 Package side

42 Nozzle

43 Nozzle slot

44 Adjustment device

45 Package back

46 Package front

1.-14. (canceled)

15. A method for filling packages open on one side, with flowable products, in particular foodstuffs, in a filling machine comprising the steps of:
introducing hot air, drying air and/or sterilising agent from above via at least one nozzle into the packages open on one side, which are transported past under the nozzle; and
directing the hot air, the drying air and/or the sterilising agent from at least one elongated nozzle slot, extending transverse to a transport direction of the at least one nozzle towards the packages,
wherein the nozzle slot extends obliquely to the transport direction of the packages.

16. The method according to claim 15, further comprising the step of:
directing the hot air, the drying air and/or the sterilising agent in a direction of the packages open on one side via an elongated nozzle slot, which extends transverse to the transport direction, over at least substantially the entire width of the packages transported past underneath.

17. The method according to claim 15, further comprising the step of:
extending the nozzle slot obliquely to the transport direction of the packages at an angle between 25° and 65°.

18. The method according to claim 15, further comprising the step of:
emerging the drying air and/or the sterilising agent out of the at least one nozzle at least substantially perpendicular to the transport direction of the packages and/or vertically.

19. The method according to claim 15, further comprising the step of:
adjusting the flow direction of the hot air, the drying air, and/or the sterilising agent as it exits the nozzle parallel and/or transverse to the transport direction of the packages while a package is transported past under the nozzle.

20. The method according to claim 19, further comprising the step of:
adjusting the flow direction of the hot air, the drying air, and/or the sterilising agent towards the transport direction of the packages as it exists the nozzle while a package is transported past under the nozzle.

21. The method according to claim 15, further comprising the step of:
transporting the packages continuously past under the at least one nozzle.

22. A filling machine for filling packages open on one side according to claim 15, wherein at least one nozzle is provided for introducing hot air, drying air and/or sterilising agent from above into the packages open on one side, wherein a transport device is provided for transporting the packages open on one side through the filling device and under the at least one nozzle and wherein the at least one nozzle has at least one elongated nozzle slot, extending transverse to the transport direction, for directing the hot air, the drying air and/or the sterilising agent in the direction of the packages open on one side,
wherein the nozzle slot is arranged oblique to the transport direction of the packages.

23. The filling machine according to claim 22,
wherein the nozzle slot extends transverse to the transport direction of the packages at least substantially over the entire width of the packages transported past underneath.

24. The filling machine according to claim 22,
wherein the nozzle slot is arranged obliquely to the transport direction of the packages at an angle between 25° and 65°.

25. The filling machine according claim 22,
wherein the nozzle is configured to direct the hot air, the drying air and/or the sterilising agent in the direction of the packages open on one side at least substantially perpendicular to the transport direction of the packages and/or vertically.

26. The filling machine according to claim 22,
wherein the at least one nozzle is configured such that it displaces the flow direction of the hot air, the drying air and/or the sterilising agent as it exits the nozzle parallel to the transport direction of the packages, in the transport direction of the packages and/or transverse to the transport direction of the packages, while a package is transported past under the nozzle.

27. The filling machine according to claim 22,
wherein the transport device has cells and/or carriers for respectively receiving the packages open on one side and for transporting the packages open on one side past under the at least one nozzle.

28. The filling machine according to claim 22,
wherein the transport device is configured to transport the packages open on one side continuously past under the at least one nozzle.

29. The method according to claim 17 further comprising the step of:
extending the nozzle slot obliquely to the transport direction of the packages at an angle between 35° and 55°.

30. The method according to claim 17 further comprising the step of:
extending the nozzle slot obliquely to the transport direction of the packages at an angle between 41° and 50°.

31. The filling machine according to claim 24, wherein the nozzle slot is arranged obliquely to the transport direction of the packages at an angle between 35° and 55°.

32. The filling machine according to claim 24, wherein the nozzle slot is arranged obliquely to the transport direction of the packages at an angle between 41° and 50°.
